(19) United States
(12) Patent Application Publication
(10) Pub. No.: US 2019/0247204 A1
(43) Pub. Date: Aug. 15, 2019

(54) RESIN-IMPREGNATED SOCKS WITH TAILS FOR MAKING CASTS

(71) Applicants: Peter M. Graf, San Francisco, CA (US); Richard M. Stess, San Anselmo, CA (US)

(72) Inventors: Peter M. Graf, San Francisco, CA (US); Richard M. Stess, San Anselmo, CA (US)

(21) Appl. No.: 15/893,548

(22) Filed: Feb. 9, 2018

(51) Int. Cl. A61F 2/50; A61F 2/76

(52) U.S. Cl. CPC A61F 2/5046; A61F 2/80; A61F 2002/5052; A61F 2/76

(57) ABSTRACT

Provided is a resin-impregnated rolled fabric sock with one or more opposed pairs of tails for ease of applying the sock to the limb, the sock being useful for preparing casts or forms on limbs. The sock is provided rolled upon itself down the outer side from the open end to the closed end and along the long axis of the sock. The tails are incorporated into the roll, however an exposed end of each tail remains external to the roll. In an embodiment, the resin-impregnated, rolled sock is provided in a sealed moisture impermeable container. Also provided are methods of use.

### FIELD OF THE INVENTION

The present invention relates generally to apparatuses for the formation of casts on limbs. More particularly, it relates to socks used for casting on limbs.

### BACKGROUND OF THE INVENTION

Holding reduced limb fractures and joint sprains in place is typically accomplished by means of a plaster immobilization cast. The cast is typically applied by wrapping plaster-coated bandages around the limb and the hardened bandage forms the immobilization cast (See, e.g. U.S. Pat. Nos. 2,388,543; 3,913,571). Such methods may also be used in total contact casting as used to treat diabetic foot ulcers and Charcot foot disease See e.g. https://en.wikipedia.org/wiki/total_contact_casting.

Prosthetic devices, used to replace some of the function of missing limbs, are usually mounted by means of a socket affixed to the residual limb or amputation stump of a patient. Sockets are most preferably custom made to conform generally to the configuration of the residual limb so as to be capable of supporting weight in the case of a leg, or loading forces in the case of an arm. However, due to the limited approximation of the fit, the weight or loading forces are not evenly distributed over all of the topologic weight bearing surfaces of the residual limb, resulting in wearing discomfort.

A common approach to forming prosthetic sockets, for example, is to form a plaster of Paris cast of the residual limb by wrapping a fabric tape with wet plaster incorporated into the weave around the limb, and removing the cast upon drying (See, e.g. U.S. Pat. No. 8,323,353; US Pat Pub 2015/0352793). The cast is then used in the laboratory as a negative. A positive plaster of Paris cast can be made from the negative and then a residual limb prosthetic socket is built in the laboratory over the positive. However, wrapping the limb with the plaster/fabric tape can be a slow and tedious process. Furthermore, the fabric tape may have elasticity and stretchable only along the long axis of the tape. Due to the uneven contour of the limb, the wrap results in different tensions at different locations of the limb topology, even when applied by an experienced technician.

U.S. Pat. No. 5,980,576 discloses an alternative method of preparing a socket for a residual limb. A socket is cast in place, thereby eliminating the need for using the original cast as a form to make a positive upon which the socket is formed. The sock is similar to that disclosed in U.S. Pat. No. 5,228,164 (disclosing kits and methods for preparing a form for making custom shoe lasts).

U.S. Ser. No. 14/489,298 (US Pat Pub 2016/0074178) discloses methods and apparatuses for creating a casted form of a residual limb having a very smooth internal surface that precisely capture the detailed topology of the residual limb. The form can then be used to create a negative mold that in turn is used to produce a custom prosthetic device.

None of the approaches that require the application of a resin-impregnated fabric sock onto a limb address the difficulty of placing the resin-impregnated sock over the limb without unwanted stretch and distortion, nor provide the material or means for a fast and easy application of the sock onto the limb.

### SUMMARY OF THE INVENTION

Provided are resin-impregnated fabric socks with an inner side, an outer side, an open end, a closed end, and a multiplicity of tails attached to the open end. The sock is provided rolled upon itself down the outer side from the open end to the closed end and along the long axis of the sock. The tails are incorporated into the roll, however an exposed end of each tail remains external to the roll. In an embodiment, the resin-impregnated, rolled sock is provided in a sealed moisture impermeable container.

The advantage of the tails becomes apparent in the method of using the invention. The invention is useful for applying an immobilization or total contact cast to a limb or making a casted form of a residual limb. The invention utilizes the rolled resin-impregnated sock, and comprises: a) removing the rolled resin impregnated sock from the moisture impermeable container; b) dipping the rolled sock in water to activate the hardening process; c) placing the inner side of the closed end of the sock against the liner at the end of the limb distal from the torso; d) evenly pulling the exposed ends of all tails up the limb thereby unrolling the sock along the limb; e) applying water to the unrolled sock, thereby accelerating the hardening of the resin; f) removing the tails from the unrolled sock.

### DESCRIPTION OF THE DRAWINGS

FIG. 1 is a perspective of the rolled sock with one pair of tails.

FIG. 2A shows the sock with one pair of tails partially unrolled.

FIG. 2B shows the sock with two pair of tails partially unrolled.

FIG. 3 shows the sock fully unrolled.

FIG. 4 is a detail of the top of the sock with price tag fasteners used to attach the tail to the sock.

FIG. 5A shows the sectional plane for FIG. 5B.

FIG. 5B is a sectional view of the rolled sock through A-A.

FIG. 6A shows the sock with tails partially applied over a liner to a residual limb.

FIG. 6B shows the sock fully unrolled and with tails removed.

FIG. 6C shows the sock with tails partially applied over a liner to a leg.

FIG. 6D shows the sock on a leg fully unrolled and with tails removed.

### DETAILED DESCRIPTION OF THE INVENTION

As used herein the terms “shell” and “form” are used interchangeably to mean the resin-hardened fabric formed around a limb.

As used herein the term “cast”, used as a noun means a shell and used as a verb means the method by which a resin-impregnated sock is used to create a cast of a residual limb or an immobilizing shell around a limb.

As used herein the term “hardened” means resin-impregnated fabric in which the resin has set or polymerized, such that it is sufficiently rigidified in its conformed shape to retain its shape upon the limb or upon removal from the limb.

As used herein the terms “hardening”, “curing”, “activating” and “setting” refer to catalyzing the polymerization of water-curable resin by the application of water to the resin.

As used herein in conjunction with fabric, the term “elastic” and “elastomeric” means elongating when placed under tension, and returning to the original configuration when tension is released.

As used herein with regard to materials and fabrics, including elastic fabrics, “stretchable” means able to increase in a dimension in response to tension, usually resulting in a contraction in a second dimension. Some material or fabric is stretchable omni-directionally in at least two dimensions (x and y), wherein lengthening the fabric in either dimension (x) will contract the fabric in the second dimension (y).

As used herein, a “sock” is a fabric in a generally tubular configuration and closed at one end. The “proximal” end of the sock is understood to refer to the open end. When a sock is applied to a patient, the proximal end is closest to the torso of the patient. In the present invention, socks are provided in a configuration in which the sock is rolled externally upon itself.

As used herein the term limb refers to an arm or leg extremity and includes the hand or foot.

As used herein the terms “residual limb” and “stump” are used interchangeably to mean the portion of an arm or leg that remains attached to a patient's torso after accidental or intentional amputation.

As used herein the term “composite” refers to a resin layup in which the resin is impregnated in a fabric, generally a woven or knit synthetic fabric.

As used herein the term “tail” refers to a long narrow attachment of flexible material.

As used herein, the term “liner” refers to material placed over a limb prior to application of a resin-impregnated sock. The liner may serve to protect a patient's skin from the resin of the sock, to cushion, pad, or lend comfort to the resulting cast, or both.

A mil is equivalent to one thousandth of an inch and 0.0254 mm.

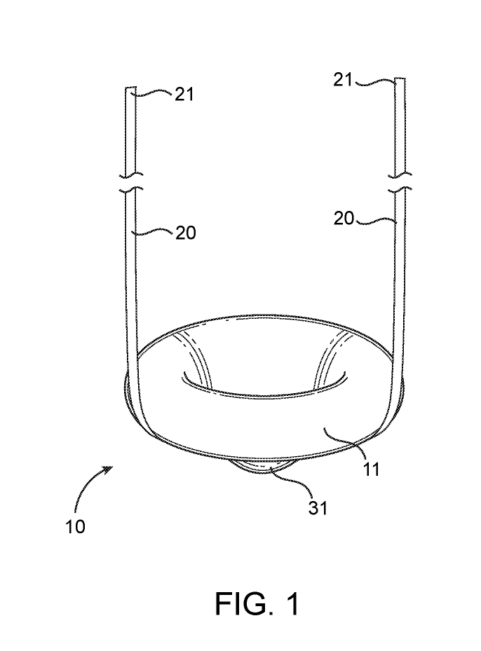

As shown in FIGS. 1-3, the present invention describes a sock [10], provided in a configuration wherein the sock is rolled upon itself [11] and impregnated with a pre-polymerized resin, and used in a method to create a custom-fitted shell on a limb. A novel feature of the sock is a multiplicity of tails [20] affixed around the perimeter of the open end [30] of the sock and incorporated into the roll of the sock to assist the practitioner in the unrolling of the sock upon the limb. The rolled fabric sock of the invention is configured for providing a cast on a limb of a subject. The sock comprises a water-curable resin impregnated into the fabric, the sock having an inner side, an outer side an open end, a closed end, and a central axis, the sock additionally comprising a multiplicity of tails affixed at the open end of the sock, and equidistant from each other around the open end of the sock, the sock rolled upon itself on the outer side and along the central axis from the open end to the closed end, thereby incorporating the tails into the roll of the sock and such that an exposed end of each tail remains external to the roll.

The fabric sock component serves to provide structure and reinforce the sock/resin composite provided, and this role is fundamentally one of increasing the mechanical properties of the sock/resin system. The mechanical properties of the fiber/resin composite are therefore dominated by the contribution of the fiber to the composite. The four main factors that govern the fiber's contribution are: the basic mechanical properties of the fiber itself; the surface interaction of fiber and resin (the ‘interface’); the amount of fiber in the composite (‘Fiber Volume Fraction’); and, the orientation of the fibers in the composite. An additional factor is the smoothness of the fabric and its draping ability. Drape is the ability of a fabric to conform to a fine, complex surface. Smoothness means a fabric surface without variability, for example, without ribbing. For woven fabric, surface smoothness and drape of a fabric are controlled primarily by the weave style. The fabric may be knitted or woven. Knitted fabric naturally provides an elastic or neck down quality when placed under tensioned. Woven fabric is usually provided with an elastic fiber such as spandex woven along with the higher modulus fiber. Alternatively, two very thin layers may be used, with the grains of the two fabrics running in different directions, thereby providing a laminate strength. If woven fabric is used, twill and satin weaves provide the greatest degree of dmpe and smoothness. In an embodiment, the fabric comprising the sock is knitted polyester with a fiber thickness of 150 denier times 2 or times 3.

To prepare the resin impregnated sock of the present invention, a dry sock is impregnated with a water-curable prepolymeric resin that is impregnated into the fabric, rolled as described and typically provided for use in a sealed moisture-impervious envelope. In an embodiment the prepolymeric resin is an aromatic isocyanate, preferably polymethylene polyphenylisocyanate (NCFI Polyurethanes, Mt. Airy, N.C.). Alternatively, other isocyanates may be used. For instance, 9910-A, Detack Natural with Apple (Bayonne Urethane Systems, St. Louis, Mo.); or Chemthane 1507/1512 (Chemline, Inc. St. Louis Mo.); or CLT-652 (Precision Performance Coatings, Mooresville, N.C.).

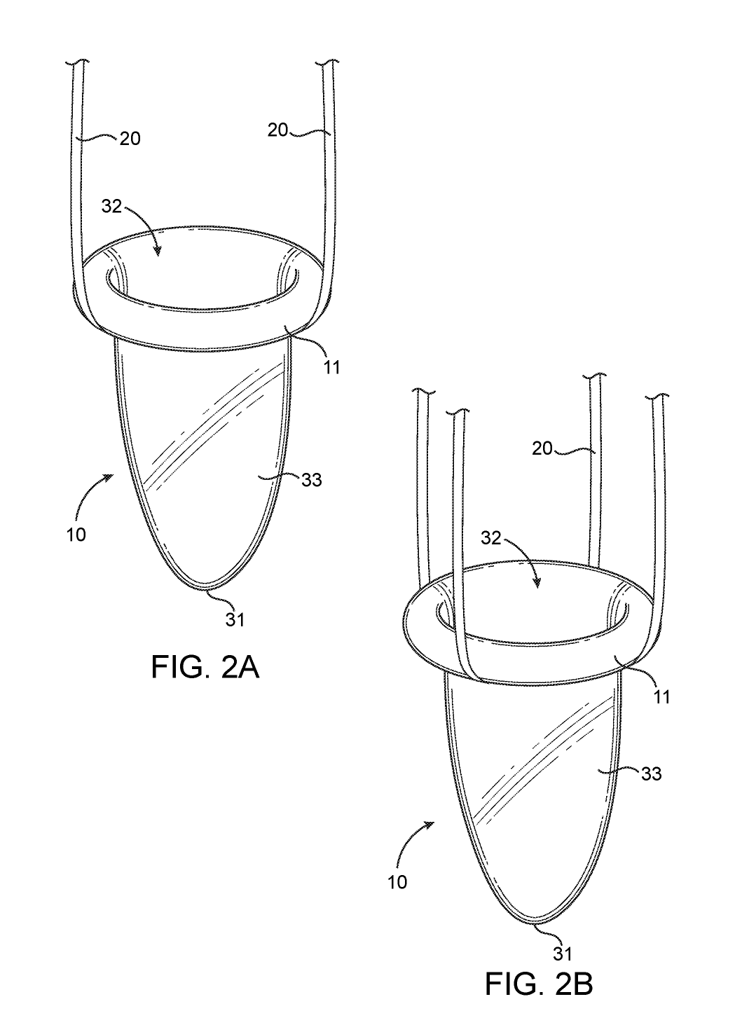

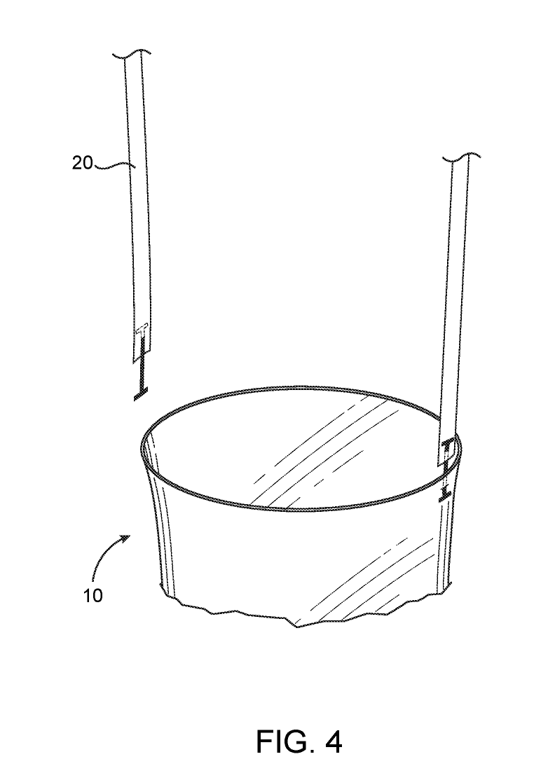

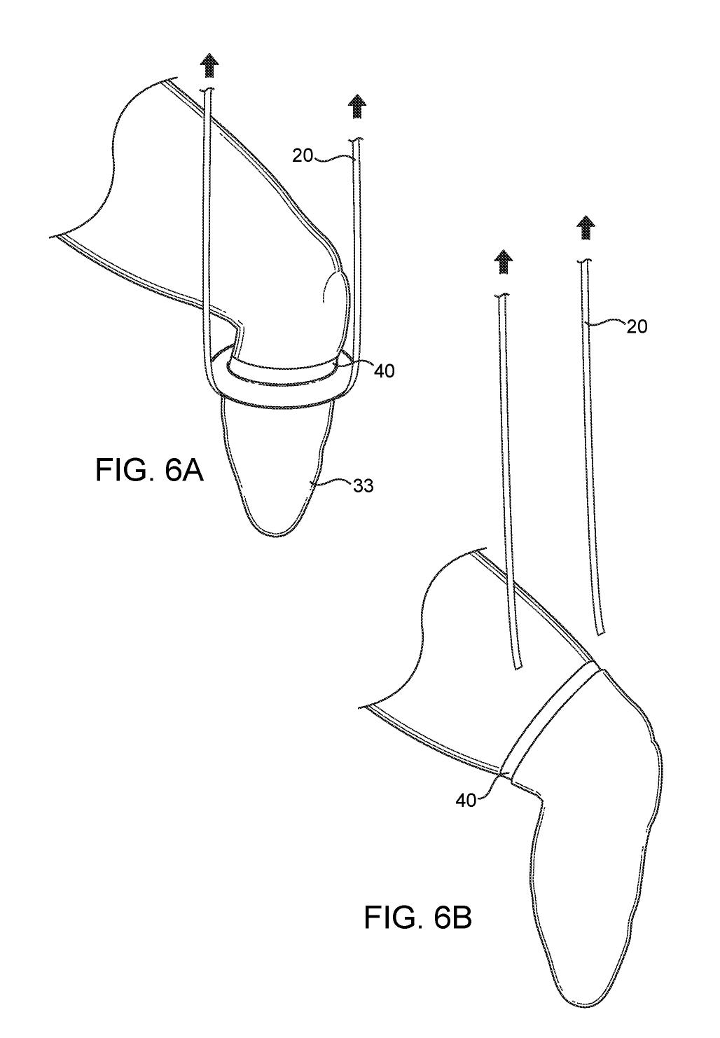

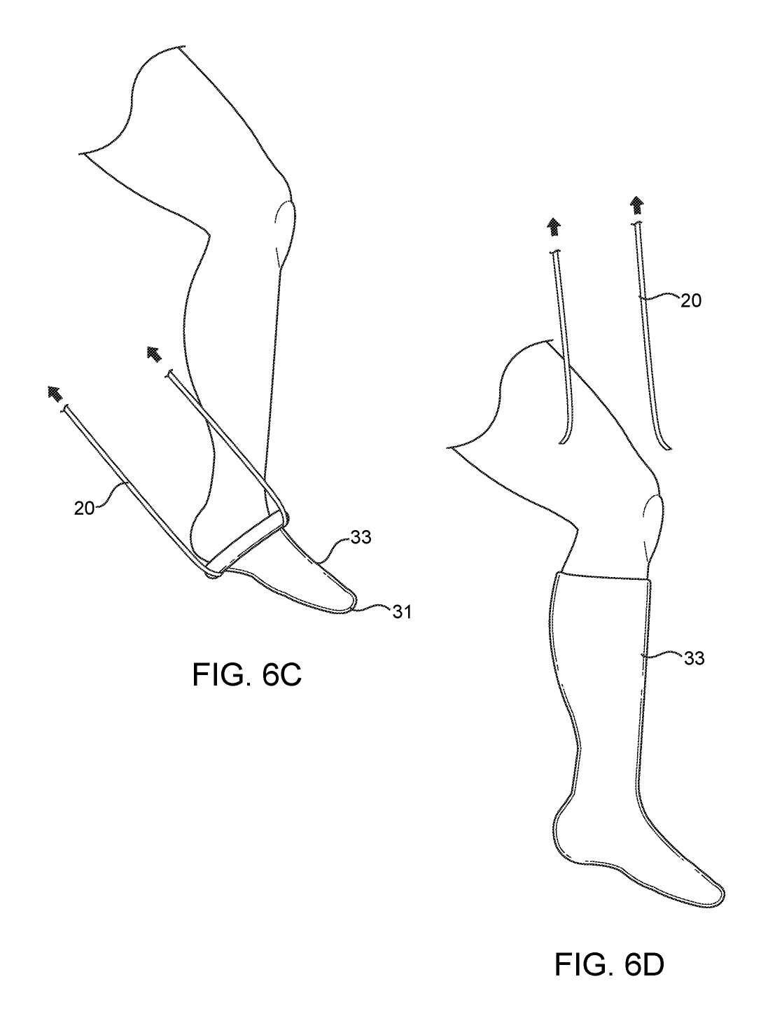

As shown in FIGS. 2-4, the multiplicity of tails [20] is affixed proximate to the open end [30] of the sock in one or more diametrically opposed pairs. The spacing of the attachment of each tail from its neighbor is equidistant or about equidistant. Each tail may be removably affixed by tacking the tail to the sock with thread, by looping or weaving the tail through the knit or weave of the sock, by application of an adhesive between the tail and the fabric, or by other means known to those in the art. In a preferred embodiment as shown in FIG. 4, plastic price tag fasteners [23] are used to connect the tail to the sock. These can be attached using a price tag gun. See, e.g. www.uline.com/b1_5653/standard-price-tag-gun-fasteners. As shown in FIG. 6, the affixation of the tail to the fabric is removable and subsequent to unrolling, the tails may be removed (see below). The tails may be fabric, made of cotton, nylon, polyester, polyethylene, Dacron, or any combination thereof. The tails may be ribbon or thread in shape. The tails may be woven or monofilament.

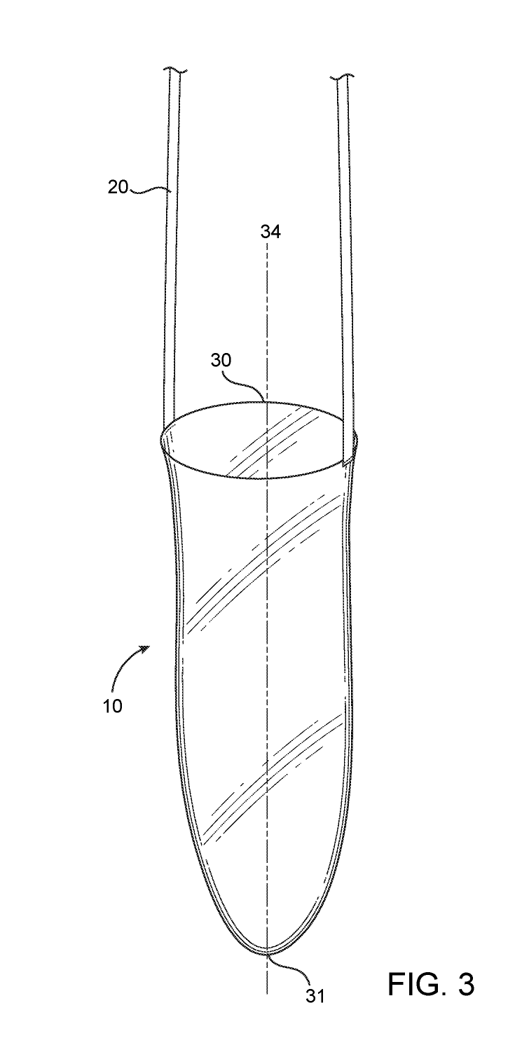

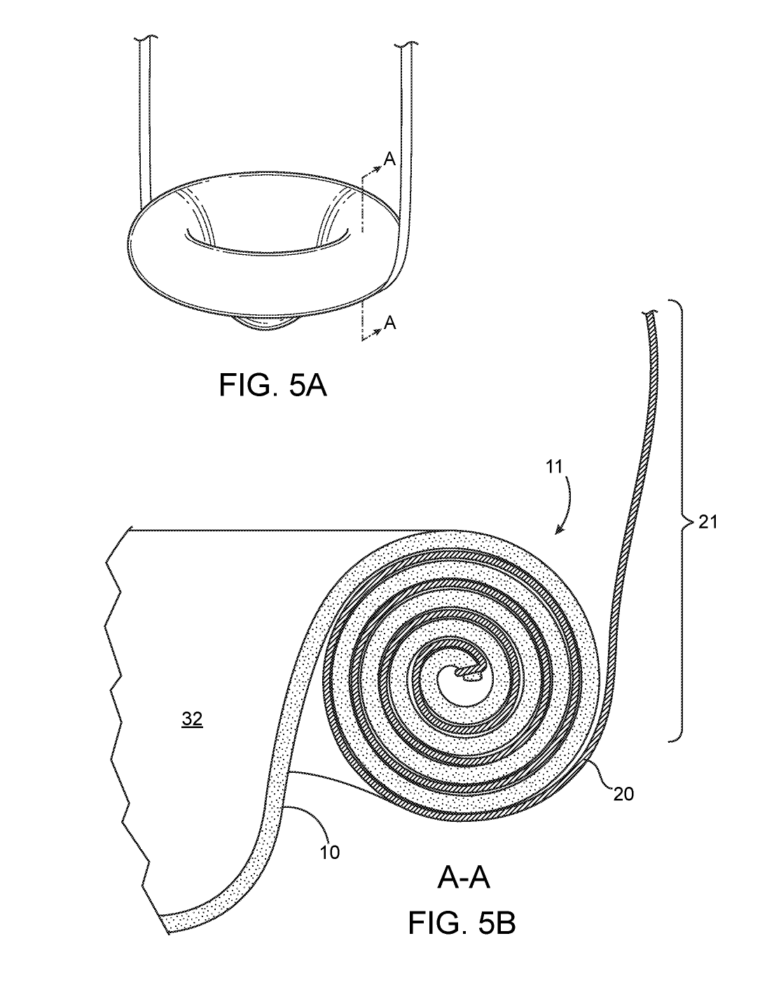

As shown in FIGS. 2 and 3, the prepolymerized resin-impregnated sock has an inner side [32], an outer side [33], an open end [30], a closed end [31], and a multiplicity of tails attached to the open end. As shown in FIGS. 3 and 5, the sock is provided rolled upon itself down its outer side from the open end [30] toward the closed end [31] and along the central axis [34] of the sock (as shown in FIG. 3). As the sock is rolled upon itself, the tails [20] are incorporated into the fabric roll of the sock [11], however an exposed end [21] of each tail remains external to the roll. In an embodiment, the resin-impregnated, rolled sock is provided in a sealed moisture impermeable container.

In an embodiment, when the resin-impregnated sock is used to make an immobilization cast on a limb, or form of a residual limb, a liner [40] is first placed upon the limb. This liner is intended either to protect the limb from the resin or to cushion, pad, or insulate the limb.

In an embodiment, the liner is a thin pliable plastic film that serves to protect the skin of the patient from the resin. The thin film liner has a generally cylindrical configuration, closed at one end distal from the torso of the patient, and typically made of polyethylene but polypropylene, poly caprolactone, polyvinyl alcohol, polyactic acid, and the like, or other polymeric material may be used. The thickness of this thin liner is no more than 1 mil (0.0254 mm) and may be as thin as 0.5 mil. In a preferred embodiment, the thickness of the thin liner is 0.7 mil. The diameter of the liner may be the same along its long axis, tapered toward the closed end, or contoured to more carefully fit the shape of various residual limbs. Similarly, liners may be provided in various diameter sizes to reduce excess liner material. The distal end of the liner, furthest from the torso of the patient, is closed, either with a flat seal of with a circular end piece.

In other embodiments the liner serves to provide cushioning, padding or comfort to the wearer, and may be made of cotton, silicone, Dacron, polyester, polyurethane, copolymer, neoprene, or combination thereof. These liners are typically from 1 to 10 mm thick. See, e.g., http://www.ottobokus.com/prosthetics/info-for-new-amputees/prosthetics-101/how-liners-work/. When the resin impregnated sock is used as an immobilization cast, typically the undercast padding comprises a cloth fabric such as a Webril™ undercast padding (See e.g., www.medline.com/product/WEBRIL-Cast-Padding-by-Covidien/Undercast-Padding/Z05-PF32741?que_1) or a cloth fabric dry sock

### METHODS OF USE

Provided are methods of making a casted form of a residual limb or an immobilization cast on a limb of a subject with a torso that utilizes the rolled resin-impregnated sock of the present invention. The method of use comprises: a) removing a rolled fabric sock from a moisture impermeable container, the sock comprising a water-curable resin impregnated into the fabric, the sock having an inner side, an outer side an open end, a closed end, and a central axis, the sock additionally comprising a multiplicity of tails affixed at the open end of the sock, and equidistant from each other around the open end of the sock, the sock rolled upon itself on the outer side and along the central axis from the open end to the closed end, thereby incorporating the tails into the roll of the sock and such that an exposed end of each tail remains external to the roll; b) placing the inner side of the closed end of the sock against the end of the limb distal from the torso of the subject; c) evenly pulling the exposed ends of the tails up the limb thereby unrolling the sock along the limb; and, d) removing the tails. In an embodiment the removed, rolled sock is dipped in water prior to placement on the limb to activate the hardening of the resin. In another embodiment, the sock is sprayed with water after placement on the limb to accelerate hardening. If a liner is used, the liner is placed on the limb prior to placing the resin impregnated sock, and the sock is unrolled over the liner.

Also provided is a kit for preparing a solid cast upon a limb of a subject, the kit comprising a resin impregnated rolled fabric sock configured for providing a cast on a limb, the sock comprising a water-curable resin impregnated into the fabric, the sock having an inner side, an outer side an open end, a closed end, and a central axis, the sock additionally comprising a multiplicity of tails affixed to the sock at the open end, equidistant from each other around the open first end of the sock, the sock rolled upon itself on the outer side and along the central axis from the open end to the closed end, thereby incorporating the tails into the roll of the sock such that an exposed end of each tail remains external to the roll, the sock provided in a sealed moisture impermeable container.

When supplied as a kit, the kit may include a set of instructions for proper use. The kit may also include a liner. If a liner is included in the kit, the practitioner first removes the liner from the kit and places or applies the liner around the limb. The practitioner then removes the moisture impermeable container that contains the pre-polymerized resin impregnated sock from the kit, opens the container and removes the rolled sock. Typically, the practitioner dips the rolled sock in warm water to activate hardening of the resin, and then places the inner side of the closed end of the sock against the liner at the distal end of the limb. The practitioner then grabs the exposed ends of the tails and evenly pulls the tails proximately along the limb as shown in FIG. 6, thereby unrolling the sock onto the limb. Finally, the practitioner removes the tails as shown in FIGS. 6 B&D This results in the pre polymerized resin impregnated sock being quickly and evenly distributed along and around the limb.

The polymerization process may be accelerated by spraying water onto the sock that has been placed upon the residual limb. Preferably warm water, 35°-42° C., is used.

When used for the preparation of prosthetics for residual limbs, the cast made thereby can be adapted for use as a socket for a residual limb as described for example in U.S. Pat. No. 5,980,576, or the cast can be adapted for use as a positive or form, useful to create a negative mold that in turn is used to make a custom prosthetic device, as described for example in US Pat Pub 2016/0074178.

In the foregoing, the present invention has been described with reference to suitable embodiments, but these embodiments are only for purposes of understanding the invention and various alterations or modifications are possible so long as the present invention does not deviate from the claims that follow.

What is claimed is:

1. A rolled fabric sock configured for providing a cast on a limb of a subject, the sock comprising a water-curable resin impregnated into the fabric, the sock having an inner side, an outer side an open end, a closed end, and a central axis, the sock additionally comprising a multiplicity of tails affixed at the open end of the sock, and equidistant from each other around the open end of the sock, the sock rolled upon itself on the outer side and along the central axis from the open end to the closed end, thereby incorporating the tails into the roll of the sock and such that an exposed end of each tail remains external to the roll.

2. The rolled fabric sock of claim 1 provided within a sealed moisture impermeable container.

3. The rolled fabric sock of claim 1 wherein the tails are cloth.

4. The rolled fabric sock of claim 1 where each tail is affixed to the sock by a monofilament line there between.

5. The rolled fabric sock of claim 1 comprising two opposing tails.

6. The rolled fabric sock of claim 1 comprising two pair of opposing tails.

7. The rolled fabric sock of claim 1 wherein the resin comprises an aromatic isocyanate.

8. The rolled fabric sock of claim 7 wherein said resin comprises polymethylene polyphenylisocyanate.

9. A method of preparing a cast on a limb of a subject with a torso comprising:
a. Removing the rolled resin impregnated fabric sock of claim 2 from the moisture impermeable container;
b. Placing the inner side of the closed end of the sock against the end of the limb distal from the torso of the subject;
c. Evenly pulling the exposed ends of all tails proximately up the limb thereby unrolling the sock along the limb; and,
d. Removing the tails from the sock.

10. The method of claim 9 additionally comprising dipping the sock in water subsequent to removing the sock from the moisture impermeable container and prior to placing the sock against the limb.

11. The method of claim 9 additionally comprising placing a liner on the limb prior to removing the sock from the container.

12. The method of claim 9 wherein the resin comprises an aromatic isocyanate.

13. The method of claim 10 wherein said resin comprises polymethylene polyphenylisocyanate.

14. A kit for preparing a solid cast upon a limb of a subject, the kit comprising a resin impregnated rolled fabric sock configured for providing a cast on a limb, the sock comprising a water-curable resin impregnated into the fabric, the sock having an inner side, an outer side an open end, a closed end, and a central axis, the sock additionally comprising a multiplicity of tails affixed to the sock at the open end, equidistant from each other around the open first end of the sock, the sock rolled upon itself on the outer side and along the central axis from the open end to the closed end, thereby incorporating the tails into the roll of the sock such that an exposed end of each tail remains external to the roll, the sock provided in a sealed moisture impermeable container.

15. The kit of claim 14 additionally comprising instructions for use.

16. The kit of claim 14 additionally comprising a liner.
